(19) United States
(12) Patent Application Publication
(10) Pub. No.: US 2003/0105334 A1
(43) Pub. Date: Jun. 5, 2003

(54) Method for preparing benzenesulfonyl compounds

(76) Inventors: Leo J. Letendre, Manchester, MO (US); Sastry A. Kunda, Chesterfield, MO (US); Donald J. Gallagher, Wheeling, IL (US); Lisa M. Seaney, Glen Ellyn, IL (US); Kathleen McLaughlin, Arlington Heights, IL (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/263,407

(22) Filed: Oct. 2, 2002

Related U.S. Application Data
(60) Provisional application No. 60/326,677, filed on Oct. 2, 2001.

(51) Int. Cl. C07D 261/06

(52) U.S. Cl. 548/247

(57) ABSTRACT

The present disclosure provides a method for the preparation of aromatic sulfonyl halides by contacting a substituted phenyl compound with a halosulfonic acid and trifluoroacetic acid. The present disclosure further provides a method for the preparation of 4-[5-methyl-3-phenylisoxazol-4-yl]benzenesulfonamide which is useful in treating cyclooxygenase-2 related disorders.

This application claims the benefit of application Serial No. 60/326,677, filed Oct. 2, 2001

### BACKGROUND OF THE INVENTION

1. Field of the Invention

This invention relates to a method of preparing aromatic sulfonyl chlorides and isoxazolyl benzenesulfonamides. This method especially relates to a method for the preparation of valdecoxib, parecoxib, parecoxib sodium and 4-[5-methyl-3-phenylisoxazol-4-yl]benzenesulfonyl chloride.

2. Description of Related Art

Substituted isoxazolyl compounds useful in treating inflammation are described in U.S. Pat. No. 5,633,272. Methods for preparing substituted isoxazol-4-yl benzenesulfonamide compounds are described in U.S. Pat. No. 5,859,257. Methods for preparing prodrugs of COX-2 inhibitors are described in U.S. Pat. No. 5,932,598. Ullmann's Encyclopedia of Industrial chemistry, 5^{th} Edition Vol. A3 page 513 describes the preparation of aromatic sulfonyl chlorides using excess chlorosulfonic acid. Ullmann's Encyclopedia also describes the preparation of aromatic sulfonamides from aromatic sulfonyl chlorides.

In the chlorosulfonation reaction, secondary reactions such as sulfone formation and poly-chlorosulfonation may be minimized with the use of large excesses of chlorosulfonic acid, by diluting with a solvent, or adding sulfone formation inhibiting substances as described in U.S. Pat. No. 5,136,043. Addition of extra chlorinating agents such as thionyl chloride (EP 115,328) complicate the process by incorporating additional operations and complicating waste handling while not addressing reactivity issues due to insolubility of the reactants. The use of chlorinated solvents such as carbon tetrachloride, chloroform or methylene chloride, while partially addressing some solubility concerns, complicate the operation of the process by creating a two phase reaction mass, generate employee exposure concerns due to the volatility and toxic nature of these solvents and further introduce these chlorinated solvents to the waste streams. Japanese patent application number JP06-145227 describes the reaction of high-density polyethylene (HDPE) with sulfuryl chloride in trifluoroacetic acid in the presence of AIBN (radical generator) to give chlorosulfonated polyethylene which is used in rubber manufacture.

### SUMMARY OF THE INVENTION

The on-going work in the area of aromatic sulfonamide synthesis and the utility of isoxazolylbenzenesulfonamide compounds in treating inflammation points to the continuing need for economical, practical and environmentally acceptable methods to prepare these compounds.

The present invention provides a novel method of preparing aromatic sulfonyl halide compounds generally and the corresponding isoxazolylbenzenesulfonamide compounds, N-[[4-(3-phenylisoxazol-4-yl)phenyl]sulfonyl]propanamide compounds and N-[[4-(3-phenylisoxazol-4-yl)phenyl]sulfonyl]propanamide, sodium salt compounds. Among the several embodiments of the present invention may be noted the provision of a process for the preparation of aromatic sulfonyl halide compounds; the provision of a process for preparing [isoxazol-4-yl]benzenesulfonamide compounds, N-[[4-(3-phenylisoxazol-4-yl)phenyl]sulfonyl]propanamide compounds and N-[[4-(3-phenylisoxazol-4-yl)phenyl]sulfonyl]propanamide, sodium salt compounds. In one embodiment the present invention provides a method of preparing an [isoxazol-4-yl]benzenesulfonamide compound having the structure of Formula 1:

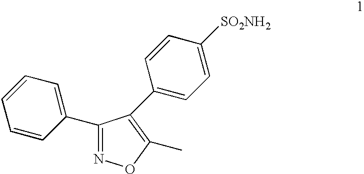

wherein the method comprises contacting a precursor compound selected from the group consisting of Formula 2 and Formula 3:

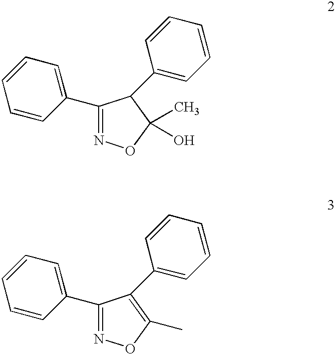

with a halosulfonic acid in the presence of trifluoroacetic acid to produce a halosulfonated product; and contacting the halosulfonated product with a source of ammonia to produce the [isoxazol-4-yl]benzenesulfonamide compound having the structure of Formula 1 (valdecoxib).

In another embodiment the present invention provides a method of preparing an N-[[4-(3-phenylisoxazol-4-yl)phenyl]sulfonyl]propanamide having the structure of Formula 1a (parecoxib)

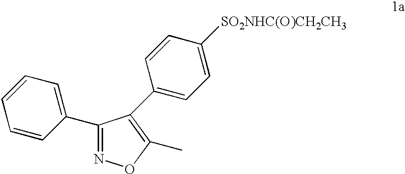

wherein the method comprises contacting a precursor compound selected from

the group consisting of Formula 2 and Formula 3 with a halosulfonic acid in the presence of trifluoroacetic acid to produce a halosulfonated product; and contacting the halosulfonated product with a source of ammonia to produce the [isoxazol-4-yl]benzenesulfonamide; and contacting the sulfonamide with a propionating agent to produce the N-[[4-(3-phenylisoxazol-4-yl)phenyl]sulfonyl]propanamide compound having the structure of Formula 1a.

In another embodiment the present invention provides a method of preparing an N-[[4-(3-phenylisoxazol-4-yl)phenyl]sulfonyl]propanamide sodium salt having the structure of Formula 1b (parecoxib sodium)

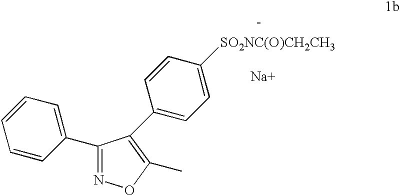

wherein the method comprises contacting a precursor compound selected from

the group consisting of Formula 2 and Formula 3 with a halosulfonic acid in the presence of trifluoroacetic acid to produce a halosulfonated product; and contacting the halosulfonated product with a source of ammonia to produce the [isoxazol-4-yl]benzenesulfonamide; and contacting the sulfonamide with a propionating agent to produce the N-[[4-(3-phenylisoxazol-4-yl)phenyl]sulfonyl]propanamide; and contacting the propanamide with a sodium base to produce the N-[[4-(3-phenylisoxazol-4-yl)phenyl]sulfonyl]propanamide, sodium salt compound having the structure of Formula 1b.

In another embodiment the present invention provides a method of preparing an N-[[4-(3-phenylisoxazol-4-yl)phenyl]sulfonyl]sulfonamide having the structure of Formula 1, wherein the method comprises forming a diphenylethanone oxime derivative compound by contacting a 1,2-diphenylethanone with a source of hydroxylamine; and contacting said oxime compound with a strong base and an acetylating agent to form a diphenylisoxazoline derivative compound; and contacting the diphenylisoxazoline derivative compound with trifluoroacetic acid and a halosulfonic acid to form a halosulfonated product; and contacting the halosulfonated product with a source of ammonia to produce the [isoxazol-4-yl]benzenesulfonamide compound having the structure of Formula 1.

In another embodiment the present invention provides a method of preparing an N-[[4-(3-phenylisoxazol-4-yl)phenyl]sulfonyl]propanamide of Formula 1a, wherein the method comprises forming a diphenylethanone oxime derivative compound by contacting a 1,2-diphenylethanone with a source of hydroxylamine; and contacting said oxime derivative compound with a strong base and an acetylating agent to form a diphenylisoxazoline derivative compound; and contacting the diphenylisoxazoline derivative compound with trifluoroacetic acid and a halosulfonic acid to form a halosulfonated product; and contacting the halosulfonated product with a source of ammonia to produce the [isoxazol-4-yl]benzenesulfonamide compound having the structure of Formula 1; and contacting the sulfonamide compound with a propionating agent to produce the N-[[4-(3-phenylisoxazol-4-yl)phenyl]sulfonyl]propanamide compound having the structure of Formula 1a.

In another embodiment the present invention provides a method of preparing an N-[[4-(3-phenylisoxazol-4-yl)phenyl]sulfonyl]propanamide, sodium salt compound having the structure of Formula 1b, wherein the method comprises forming a diphenylethanone oxime derivative compound by contacting a 1,2-diphenylethanone with a source of hydroxylamine; contacting said oxime derivative compound with a strong base and an acetylating agent to form a diphenylisoxazoline derivative; contacting the diphenylisoxazoline derivative with trifluoroacetic acid and a halosulfonic acid to form a halosulfonated product; contacting the halosulfonated product with a source of ammonia to produce the [isoxazol-4-yl]benzenesulfonamide 1; contacting the sulfonamide with propionating agent to produce the N-[[4-(3-phenylisoxazol-4-yl)phenyl]sulfonyl]propanamide compound having the structure of Formula 1a; and contacting the propanamide compound with a sodium base to produce the N-[[4-(3-phenylisoxazol-4-yl)phenyl]sulfonyl]propanamide, sodium salt compound having the structure of Formula 1b.

In another embodiment the present invention provides a method of preparing a benzenesulfonyl halide compound having the structure of Formula 4:

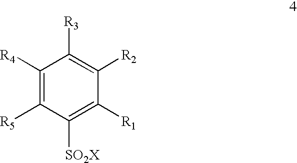

wherein X is a halogen atom and R¹, R², R³, R⁴ and R⁵ are independently selected from the group consisting of hydrogen, alkyl, alkenyl, alkynyl, cycloalkyl, aryl, heterocyclyl, alkoxy, alkylamino, alkylthio, acyl; wherein alkyl, alkenyl, alkynyl, cycloalkyl, aryl, heterocyclyl is each optionally substituted with one or more moieties selected from the group consisting of alkyl, alkenyl, alkynyl, cycloalkyl, aryl, heterocyclyl, alkoxy, alkylamino, alkylthio, acyl, halo, haloalkylaryl, alkoxyaryl, haloalkyl, and alkoxyhaloalkyl; wherein the method comprises contacting a substituted phenyl compound having the structure of Formula 5:

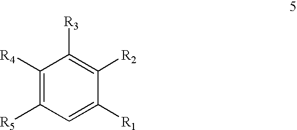

with a halosulfonic acid in the presence of trifluoroacetic acid, thereby forming a benzenesulfonyl halide compound.

In another embodiment the present invention provides a method of preparing a 5-phenylisoxazol-4-yl benzenesulfonyl halide wherein the method comprises contacting a 4,5-diphenylisoxazole compound with a halosulfonic acid in the presence of trifluoroacetic acid, thereby forming a 5-phenylisoxazol-4-yl benzenesulfonyl halide compound having the structure of Formula 6:

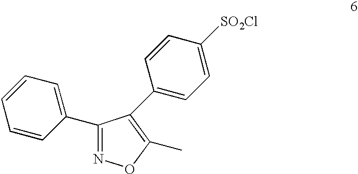

Further scope of the applicability of the present invention will become apparent from the detailed description provided below. However, it should be understood that the following detailed description and examples, while indicating preferred embodiments of the invention, are given by way of illustration only since various changes and modification within the spirit and scope of the invention will become apparent to those skilled in the art from this detailed description.

### BRIEF DESCRIPTION OF THE FIGURES

FIG. 1 shows a process by which 4-[5-methyl-3-phenylisoxazol-4-yl]benzenesulfonamide having the structure of Formula 1 can be prepared.

FIG. 2 shows the process by which the compounds having the structure of Formulae 1a and 1b can be prepared from the compound having the structure of Formula 1.

### DETAILED DESCRIPTION OF THE PREFERED EMBODIMENTS

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

The contents of each of the references cited herein, including the contents of the references cited within these primary references, are herein incorporated by reference in their entirety.

a. Definitions

The following definitions are provided in order to aid the reader in understanding the detailed description of the present invention:

“Alkyl,” “alkenyl,” and “alkynyl” unless otherwise noted are each straight chain or branched chain hydrocarbon groups of from one to about twenty carbons for alkyl or two to about twenty carbons for alkenyl and alkynyl in the present invention and therefore mean, for example, methyl, ethyl, propyl, butyl, pentyl or hexyl and ethenyl, propenyl, butenyl, pentenyl, or hexenyl and ethynyl, propynyl, butynyl, pentynyl, or hexynyl respectively and isomers thereof.

“Cycloalkyl” is a mono- or multi-ringed carbocycle wherein each ring contains three to ten carbon atoms, and wherein any ring can contain one or more double or triple bonds. Examples include radicals such as cyclopropyl, cyclobutyl, cyclopentyl, cyclohexyl, cycloalkenyl, and cycloheptyl.

“Aryl” means a fully unsaturated mono- or multi-ring carbocycle, including, but not limited to, substituted or unsubstituted phenyl, naphthyl, or anthracenyl.

“Heterocyclyl” means a saturated or unsaturated mono- or multi-ring carbocycle wherein one or more carbon atoms can be replaced by N, S, P, or O. This includes, for example, the following structures:

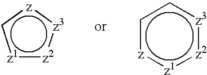

wherein Z, Z¹ Z² or Z³ is C, S, P, O, or N, with the proviso that one of Z, Z¹, Z² or Z³ is other than carbon, but is not O or S when attached to another Z atom by a double bond or when attached to another O or S atom. Furthermore, the optional substituents are understood to be attached to Z, Z¹, Z² or Z³ only when each is C. The point of attachment to the molecule of interest can be at the heteroatom or elsewhere within the ring.

The term “alkoxy” means a radical comprising an alkyl radical that is bonded to an oxygen atom, such as a methoxy radical. More preferred alkoxy radicals are “lower alkoxy” radicals having one to ten carbon atoms. Examples of such radicals include methoxy, ethoxy, propoxy, isopropoxy, butoxy and tert-butoxy.

The term “alkylamino” means a radical comprising an alkyl radical that is bonded to a nitrogen atom, such as a N-methylamino radical. More preferred radicals are “lower alkylamino” radicals having one to ten carbon atoms. Examples of such radicals include N-methylamino, N,N-dimethylamino, N-ethylamino, N,N-diethylamino, N,N-dipropylamino, N-butylamino, and N-methyl-N-ethylamino.

The term “alkylthio” means a radical comprising an alkyl radical that is bonded to a sulfur atom, such as a methylthio radical. More preferred alkylthio radicals are “lower alkylthio” radicals having one to ten carbon atoms. Examples of such radicals include methylthio, ethylthio, propylthio and butylthio.

The term “acyl” means a radical comprising an alkyl or aryl radical that is bonded to a carboxy group such as a carboxymethyl radical. More preferred acyl radicals are “carboxy lower alkyl” radicals having one to ten carbon atoms and carboxyphenyl radicals. Examples of such radicals include carboxymethyl, carboxyethyl and carboxypropyl.

The term “halo” means a fluoro, chloro, bromo or iodo group.

The term “haloalkyl” means alkyl substituted with one or more halogens. Examples of such radicals include chloromethyl, difluoromethyl, trifluoromethyl, pentafluoroethyl, dichloromethyl and trichloromethyl.

When used in combination, for example “haloalkylaryl”, “alkoxyaryl” or “alkoxyhaloalkyl” the individual terms listed above have the meaning indicated above.

As used herein, Me means methyl; Et means ethyl; Pr means propyl; i-Pr or Prⁱ each means isopropyl; Bu means butyl; t-Bu or Bu^{t} each means tert-butyl.

Weak acid is an acid of such strength to produce sufficient protonated hydroxylamine to react with a diphenylethanone compound to produce a diphenylethanone oxime derivative compound.

Strong base is a base that upon contacting an oxime derivative compound produces sufficient di-anion species to further react with an acetylating agent.

Deprotonating base is a base which reacts with a hydroxylamine salt to produce sufficient hydroxylamine to further react with a diphenylethanone compound to produce a diphenylethanone oxime derivative compound.

Propionating agent means an agent that upon contacting a benzenesulfonamide compound having the structure of Formula 1 produces a sulfonyl propanamide compound. A propionating agent can include an active ester such as a propionyl anhydride, a propionyl mixed anhydride, a propionyl thioester, a propionyl carbonates or the like. A propionating agent also includes a propionyl halide preferably propionyl chloride, an active amides such as N-propionylimidazole, N-alkyl-N-alkoxypropionamides and the like. Many more active propionating agents are described in M. Bodanszky, Principles of Peptide Synthesis 14-61 (second revised edition, Springer Verlag 1993).

An acylating agent is an agent which upon contacting a 1,2-diphenyl ethanone derivative oxime in the presence of a strong base produces an isoxazolyl compound or an isoxazole compound having the structure of Formula 2 and/or 3. Acylating agents can include an acetic anhydride, preferably diacetic anhydride. An acylating agent can also include an acyl halide, preferably acetyl chloride. An acylating agent can also include a C1 to about C6 alkyl acetate selected from the group consisting of methyl acetate, ethyl acetate, propyl acetate and butyl acetate and more preferably ethyl acetate.

A sodium base is a base which upon contacting with the benzenepropanamide compound having the structure of Formula 1a produces a sulfonyl propanamide sodium salt compound. Sodium bases can include sodium hydroxide, a sodium alkoxide such as sodium ethoxide or sodium methoxide. A sodium base can also be sodium hydride or sodium carbonate.

A protecting group is a chemical moiety which serves to protect a chemical functionality of a molecule while the molecule is undergoing a chemical reaction at a different locus in the molecule. Preferably, after the chemical reaction, the protecting group can be removed to reveal the original chemical functionality. A hydroxyl protecting group for example can protect a hydroxyl group. A protected hydroxymethyl group comprises a hydroxymethyl group in which the hydroxyl group is protected by a protecting group. Useful protecting groups can vary widely in chemistry. Numerous hydroxyl protecting groups are described in Theodora W. Greene and Peter G. M. Wuts Protective Groups in Organic Chemistry 86-97 (Third Edition, John Wiley & Sons, 1999). An example of a protected hydroxymethyl group is a deactivated benzyloxymethyl group and the like.

b. Process Details

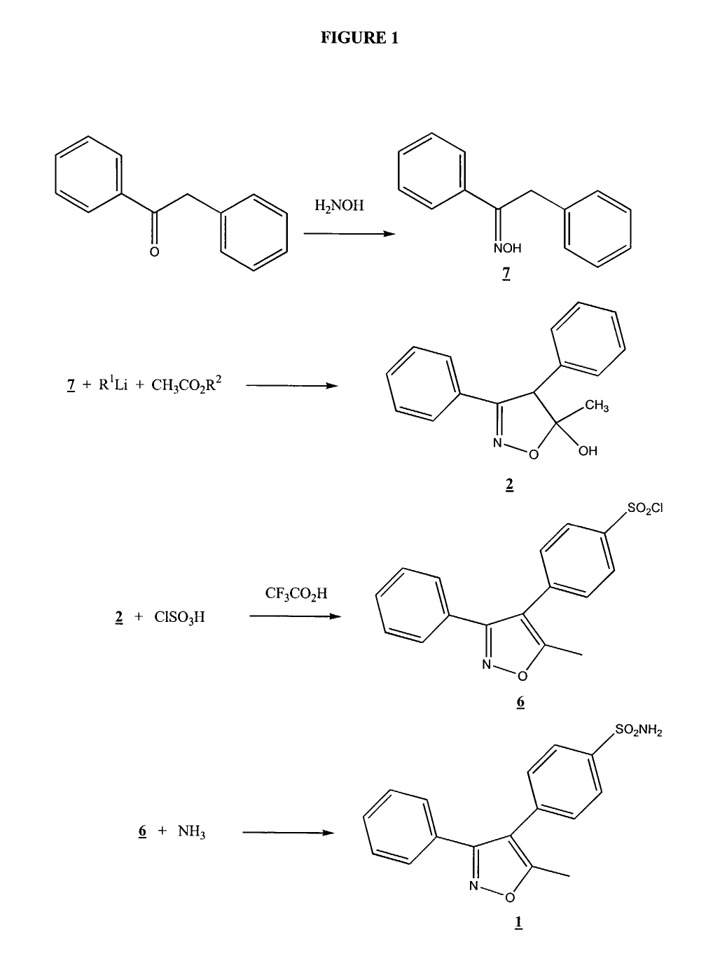

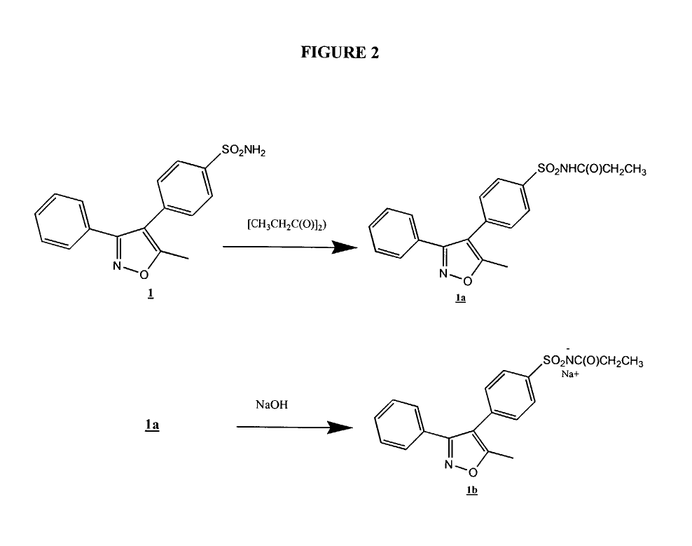

In accordance with the present invention, a process is now provided for preparing benzenesulfonyl derivatives, in particular 4-[5-methyl-3-phenylisoxazol-4-yl]benzenesulfonyl chloride having the structure of Formula 6, 4-[5-methyl-3-phenylisoxazol-4-yl]benzenesulfonamide (valdecoxib) having the structure of Formula 1, N-[[4-(5-methyl-4-phenylisoxazol-4-yl)phenyl]sulfonyl]propanamide (parecoxib) having the structure of Formula 1a and N-[[4-(5-methyl-4-phenylisoxazol-4-yl)phenyl]sulfonyl]propanamide sodium salt (parecoxib sodium) having the structure of Formula 1b. A schematic of a method for the preparation of valdecoxib using the present invention is provided in FIG. 1. A schematic of a method for the preparation of parecoxib and parecoxib sodium from valdecoxib using the present invention is provided in FIG. 2.

In one embodiment, the present invention provides a method of preparing an [isoxazol-4-yl]benzenesulfonamide compound having the structure of Formula 1 comprising contacting a precursor compound selected from the group consisting of Formula 2 and Formula 3 with a halosulfonic acid in the presence of trifluoroacetic acid to produce a halosulfonated product and contacting the halosulfonated product with a source of ammonia to produce the [isoxazol-4-yl]benzenesulfonamide compound having the structure of Formula 1. The halosulfonic acid useful in the various embodiments of the present invention, for example, can be any convenient halosulfonic acid. Preferably the halosulfonic acid is selected from the group consisting of bromosulfonic acid and chlorosulfonic acid, and more preferably chlorosulfonic acid. The source of ammonia useful in the various embodiments of the present invention, for example, can be selected from the group consisting of ammonium hydroxide and anhydrous ammonia. More preferred the source of ammonia comprises ammonium hydroxide. In another preferred embodiment, the source of ammonia comprises anhydrous ammonia.

In another embodiment, the present invention provides a method of preparing an N-[[4-(3-phenylisoxazol-4-yl)phenyl]sulfonyl]propanamide compound having the structure of Formula 1a comprising contacting a precursor compound selected from the group consisting of Formula 2 and Formula 3 with a halosulfonic acid in the presence of trifluoroacetic acid to produce a halosulfonated product and contacting the halosulfonated product with a source of ammonia to produce an [isoxazol-4-yl]benzenesulfonamide compound having the structure of Formula 1 and contacting the [isoxazol-4-yl]benzenesulfonamide compound with a propionating agent to produce an N-[[4-(3-phenylisoxazol-4-yl)phenyl]sulfonyl]propanamide compound having the structure of Formula 1a. The propionating agent useful in the various embodiments of the present invention, for example, can be selected from the group consisting of an anhydride of propionic acid, a propionyl halide, a propionyl thioester, a propionyl carbonate and an N-propionylimidazole. Preferably the propionating agent is an anhydride of propionic acid and more preferably propionic anhydride and still more preferably a propionyl halide and still more preferably propionyl chloride.

In another embodiment, the present invention provides a method of preparing an N-[[4-(3-phenylisoxazol-4-yl)phenyl]sulfonyl]propanamide, sodium salt compound having the structure of Formula 1b comprising contacting a precursor compound selected from the group consisting of Formula 2 and Formula 3 with a halosulfonic acid in the presence of trifluoroacetic acid to produce a halosulfonated product and contacting the halosulfonated product with a source of ammonia to produce an [isoxazol-4-yl]benzenesulfonamide compound having the structure of Formula 1 and contacting the [isoxazol-4-yl]benzenesulfonamide compound having the structure of Formula 1 with a propionating agent to produce an N-[[4-(3-phenylisoxazol-4-yl)phenyl]sulfonyl]propanamide compound having the structure of Formula 1a and further contacting the compound of Formula 1a with a sodium base to produce an N-[[4-(3-phenylisoxazol-4-yl)phenyl]sulfonyl]propanamide, sodium salt compound having the structure of Formula 1b. The sodium base useful in the various embodiments of the present invention, for example, is selected from the group consisting of sodium hydroxide, a sodium alkoxide, sodium hydride and sodium carbonate. Preferably the sodium base is sodium methoxide and more preferably the sodium base is sodium hydroxide.

In another embodiment the present invention provides a method of preparing an [isoxazol-4-yl]benzenesulfonamide compound having the structure of Formula 1 comprising contacting a 1,2-diphenylethanone compound with a source of hydroxylamine to form a diphenylethanone oxime derivative compound, and contacting the oxime derivative compound with a strong base and an acetylating agent to form a diphenylisoxazoline derivative and contacting the diphenylisoxazoline derivative with trifluoroacetic acid and a halosulfonic acid to form a halosulfonated product and contacting the halosulfonated product with a source of ammonia to produce an [isoxazol-4-yl]benzenesulfonamide compound having the structure of Formula 1. The source of hydroxylamine useful in the various embodiments of the present invention, for example, can be, an aqueous solution comprising hydroxylamine. Preferably the source of hydroxylamine is an aqueous solution comprising hydroxylamine and a weak acid wherein the weak acid is a carboxylic acid and preferably an alkyl carboxylic acid and still more preferably the alkyl carboxylic acid selected from the group consisting of formic acid, acetic acid and propionic acid and more preferably is acetic acid. Most preferably the source of hydroxylamine is an aqueous solution of hydroxylamine and acetic acid.

The source of hydroxylamine can also comprise a hydroxylamine salt and a deprotonating base. The hydroxylamine salt is selected from the group consisting of hydroxylamine hydrochloride, hydroxylamine sulfate and hydroxylamine acetate. The hydroxylamine salt is preferably hydroxylamine hydrochloride. The deprotonating base is selected from the group consisting of sodium hydroxide, potassium hydroxide and sodium acetate. The deprotonating base is preferably sodium acetate. Another more preferred source of hydroxylamine comprises hydroxylamine hydrochloride and sodium acetate.

The strong base which is contacted with the oxime derivative compound useful in the various embodiments of the present invention, for example, can be preferably selected from the group consisting of a lithium dialkylamide, an aryl lithium, an arylalkyl lithium and an alkyl lithium. The strong base can be a lithium dialkylamide and preferably lithium diisopropylamide. More preferably the strong base is a C₁ to about C₁₀ alkyl lithium and more preferably selected from the group consisting of butyl lithium, hexyl lithium, heptyl lithium, octyl lithium and still more preferably butyl lithium or hexyl lithium.

The acetylating agent useful in the various embodiments of the present invention, for example, can be selected from the group consisting of an alkyl acetate, an acetic anhydride, an N-alkyl-N-alkoxyacetamide and an acetyl halide. The acetylating agent can be an acetic anhydride and is preferably acetic anhydride and can be an acetyl halide and preferably acetyl chloride and more preferably a C1 to about C6 alkyl acetate selected from the group consisting of methyl acetate, ethyl acetate, propyl acetate and butyl acetate and more preferably ethyl acetate.

In another embodiment the present invention provides a method of preparing an N-[[4-(3-phenylisoxazol-4-yl)phenyl]sulfonyl]propanamide compound having the structure of Formula 1a comprising contacting a 1,2-diphenylethanone compound with a source of hydroxylamine to form a diphenylethanone oxime derivative compound; contacting the oxime derivative compound with a strong base and an acetylating agent to form a diphenylisoxazoline derivative; contacting the diphenylisoxazoline derivative with trifluoroacetic acid and a halosulfonic acid to form a halosulfonated product; contacting the halosulfonated product with a source of ammonia to produce an [isoxazol-4-yl]benzenesulfonamide compound having the structure of Formula 1; and contacting the [isoxazol-4-yl]benzenesulfonamide compound with a propionating agent to produce an N-[[4-(3-phenylisoxazol-4-yl)phenyl]sulfonyl]propanamide compound having the structure of Formula 1a.

In another embodiment the present invention provides a method of preparing an N-[[4-(3-phenylisoxazol-4-yl)phenyl]sulfonyl]propanamide, sodium salt compound having the structure of Formula 1b comprising forming a diphenylethanone oxime derivative compound by contacting a 1,2-diphenylethanone compound with a source of hydroxylamine and contacting the oxime derivative compound with a strong base and an acetylating agent to form a diphenylisoxazoline derivative and contacting the diphenylisoxazoline derivative with trifluoroacetic acid and a halosulfonic acid to form a halosulfonated product and contacting the halosulfonated product with a source of ammonia to produce an [isoxazol-4-yl]benzenesulfonamide compound having the structure of Formula 1 and contacting the [isoxazol-4-yl]benzenesulfonamide compound with a propionating agent to produce an N-[[4-(3-phenylisoxazol-4-yl)phenyl]sulfonyl]propanamide compound having the structure of Formula 1a and further contacting the compound of Formula 1a with a sodium base to produce an N-[[4-(3-phenylisoxazol-4-yl)phenyl]sulfonyl]propanamide, sodium salt compound having the structure of Formula 1b.

In another embodiment the present invention provides a method of preparing a benzenesulfonyl halide compound having the structure of Formula 4:

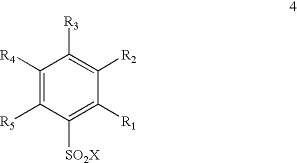

wherein X is a halogen atom and R¹, R², R³, R⁴ and R⁵ are independently selected from the group consisting of hydrogen, alkyl, alkenyl, alkynyl, cycloalkyl, aryl, heterocyclyl, alkoxy, alkylamino, alkylthio, acyl; wherein alkyl, alkenyl, alkynyl, cycloalkyl, aryl, heterocyclyl is each optionally substituted with one or more moieties selected from the group consisting of alkyl, alkenyl, alkynyl, cycloalkyl, aryl, heterocyclyl, alkoxy, alkylamino, alkylthio, acyl, halo, haloalkylaryl, alkoxyaryl, haloalkyl, protected hydroxymethyl, arylalkoxymethyl, and alkoxyhaloalkyl; wherein the method comprises contacting a substituted phenyl compound having the structure of Formula 5:

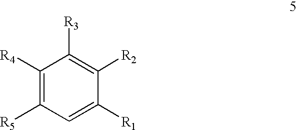

with a halosulfonic acid in the presence of trifluoroacetic acid, thereby forming a benzenesulfonyl halide compound.

More preferred embodiment of the present invention a method wherein R³ is heterocyclyl optionally substituted with one or more moieties selected from the group consisting of alkyl, alkenyl, alkynyl, cycloalkyl, aryl, heterocyclyl, alkoxy, alkylamino, alkylthio, acyl, halo, haloalkylaryl, alkoxyaryl, haloalkyl, alkoxycarbonyl, protected hydroxymethyl, arylalkoxymethyl, and alkoxyhaloalkyl; and R¹, R², R⁴ and R⁵ are hydrogen. Still further preferred is the method wherein R3 is selected from the group consisting of isoxazolyl and pyrazolyl wherein R³ is optionally substituted with one or more moieties selected from the group consisting of alkyl, alkenyl, alkynyl, cycloalkyl, aryl, heterocyclyl, alkoxy, alkylamino, alkylthio, acyl, halo, haloalkylaryl, alkoxyaryl, haloalkyl, alkoxycarbonyl, protected hydroxymethyl, arylalkoxymethyl, and alkoxyhaloalkyl; and R¹, R², R⁴ and R⁵ are hydrogen.

In another embodiment the present invention provides a method of preparing a 5-phenylisoxazol-4-yl benzenesulfonyl halide wherein the method comprises contacting a 4,5-diphenylisoxazole with a halosulfonic acid in the presence of trifluoroacetic acid, thereby forming a 5-phenylisoxazol-4-yl benzenesulfonyl halide compound having the structure of Formula 6:

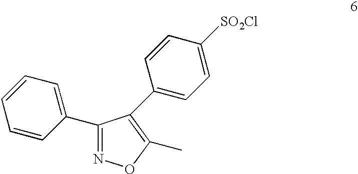

In another embodiment the present invention provides a method of preparing a 5-phenylisoxazol-4-yl benzenesulfonyl halide wherein the method comprises contacting a compound selected from the group consisting of Formula 2 and Formula 3 with a halosulfonic acid in the presence of trifluoroacetic acid, thereby forming a 5-phenylisoxazol-4-yl benzenesulfonyl halide compound having the structure of Formula 6.

As provided herein trifluoroacetic acid is a useful solvent for the halosulfonation of aromatic compounds to give the corresponding aryl sulfonyl halides. The use of trifluoroacetic acid provides solubilization of many solid substrates. The higher boiling point of trifluoroacetic acid versus methylene chloride enables the halosulfonation reaction to be carried out at higher temperatures and which can have the benefit of shorter reaction times. In addition, trifluoroacetic acid can be used to pre-dissolve the solid aromatic substrates making it easier and safer to transfer the substrate from a filtration device to a halosulfonation reactor. The use of trifluoroacetic acid also eliminates chlorinated hydrocarbons from air emissions and aqueous waste streams.

The halosulfonation reaction under which compounds 2, 3, and 5 react to form the aromatic sulfonyl chlorides of structures 4 and 6 is carried out in the presence of trifluoroacetic acid.

The ratio of trifluoroacetic acid used and reaction time can vary as shown in the table below.

| | | Reaction | | | |
|---|---|---|---|---|---|
| TFA | Temperature | time | Completion | | |
| Equivalents | time □C. | Hours (h) | time | | Valdecoxib¹ |
| 2.0 | 70 | 2 | <30 | min | 78 |
| 2.0 | 40 | 6 | 3.3 | h | 80 |
| 3.0 | 60 | 3 | 50 | min | 76 |
| 4.0 | 70 | 2.5 | 1 | h | 87 |
| 4.0 | 40 | 4 | 4 | h | 77 |

¹Endpoint mol % values from in process samples quenched with acetonitrile, water, and ammonium hydroxide mixture.

It is preferable to use sufficient trifluoroacetic acid to ensure a fluid reaction mass. For the conversion of 2 and 3 to 6, the amount of trifluoroacetic acid can range from about 1.5 to about 4 weight equivalents relative to 2 and 3. In one preferred embodiment, the weight equivalent of trifluoroacetic acid was equal to the weight of 2 and 3.

The halosulfonation reaction can proceed over a range of temperatures and preferably is performed within the range of −20° C. to 100° C. and more preferably about 30° C. to 70° C., still more preferably about 55° C. to 65° C.

The chlorosulfonation reaction can proceed at atmospheric pressure or under pressure and is preferably carried out below the boiling point of trifluoroacetic acid under atmospheric pressure. The chlorosulfonation can proceed at higher temperatures with enough pressure on the reactor system to prevent losses due to volatilization.

c. Detailed Preparative Methods

The starting materials for use in the methods of preparation of the invention are known or can be prepared by conventional methods known to a skilled person or in an analogous manner to processes described in the art. The following examples are intended to be illustrative of the many embodiments of the present invention and are not meant to be limiting in scope. Generally, the process methods of the present invention can be performed as follows. Larger scale preparation can be performed, for example, by proportionately increasing ingredient quantities.

### EXAMPLE 1

Preparation of 4-(5-Methyl-3-phenyl-4-isoxazolyl)benzenesulfonamide (valdecoxib, 1)

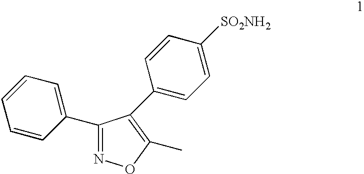

Step 1: Preparation of 1,2-Diphenylethanone, oxime 7.

To a solution of deoxybenzoin (2.3 kg, 11.7 mol), acetic acid (669 mL, 11.7 mol), and ethanol 3A (8.05 L, 190 proof) at 70° C. was added 50 weight percent hydroxylamine (800 mL, 13.3 mol) via an addition funnel. The addition funnel was rinsed with water (460 mL) and the reaction mixture held at 70° C. for 1 hour. The reaction was monitored for reaction completion by HPLC. Water was charged to the reactor (2.87 L) and the temperature reduced to 50° C. An aliquot (250 mL) was removed from the reactor, cooled, and allowed to crystallize. This mixture was reintroduced into the reactor to seed the batch and initiate crystallization. Seeding is not necessary, but, if used, helps increase the bulk density of the oxime product thereby enhancing the handling properties of the resulting oxime. After stirring for 1 hour, water (8.78 L) was added over 2.5 hours and the mixture cooled to 20° C. The mixture was pressure filtered; and the cake was washed with 2:1 Water/ethanol 3A (10.8 L), and then water (4.5 L). The cake was blown dry with N₂ overnight to afford a white solid (2.34 kg, 95% yield, 96:4 E/Z oxime isomers). High-resolution MS (ES) m/z (M+H)⁺ calculated: 212.1075; found 212.1085.

Step 1 (alternate procedure) Preparation of 1,2-Diphenylethanone, oxime 7.

To a solution of deoxybenzoin (75.0 g, 0.382 mole), sodium acetate (34.5 g, 0.420 mole), and ethanol 3A (267 mL, 190 proof) at 70° C. was added 35 weight percent hydroxylamine hydrochloride (72.0 mL, 0.420 mole) via a syringe pump. The reaction mixture held at 70° C. for 1 hour and was monitored for reaction completion by HPLC. Water was charged to the reactor (75.0 mL) and the temperature reduced to 50° C. An aliquot (0.5 mL) was removed from the reactor, cooled, and allowed to crystallize. This mixture was reintroduced into the reactor to seed the batch and initiate crystallization. Seeding is not necessary, but, if used, helps increase the bulk density of the oxime product thereby enhancing the handling properties of the resulting oxime. After stirring for 1 hour, water (274 mL) was added over 1 hour and the mixture cooled to 20° C. The mixture was filtered; and the cake was washed with 2:1 Water/ethanol 3A (188 mL), and then water (100 mL). The cake was dried in a vacuum oven at 50° C. for 16 h to afford a white solid (76.39 g, 95% yield, 97:3 E/Z oxime isomers).

Step 2: Preparation of 4,5-Dihydro-5-methyl-3,4-diphenyl-5-isoxazolol, 2.

To a 500 mL jacketed reactor equipped with a mechanical stirrer, thermocouple, and positive pressure nitrogen inlet was charged 1,2-diphenyl-ethanone, oxime (31.4 grams). Tetrahydrofuran (THF) (160 mL) was added while stirring to dissolve the solid. The reaction was cooled using a jacket temperature of −15° C. n-Hexyllithium in hexanes (131 mL, 2.3 M) was charged to the reaction vessel while keeping the temperature below 10° C. After addition was complete, the mixture was stirred for 30 minutes using a jacket temperature of −15° C. Ethyl acetate (120 mL) was added keeping the temperature below 10° C. The reaction mixture was then transferred via cannula to a mixture of sodium chloride (14.0 g) in water (160 mL) that was cooled to 5° C. The reaction vessel was rinsed with 40 mL THF and this mixture was transferred to the quench flask. The quench mixture was warmed to 20° C. and the layers were separated. The organic layer was washed with a sodium bicarbonate (NaHCO₃) solution (9.6 g NaHCO₃/160 mL water). Toluene (120 mL) was added to the organic layer and the mixture was distilled until a pot temperature of 90.2° C. was attained. Heptane (439 mL) was added and the mixture was cooled at 0.5° C./min to 5° C. during which time crystals formed. The mixture was filtered through polypropylene mesh and the solid cake was washed with 100 mL of 50:50 (volume/volume) heptane:toluene. The solid was dried in a vacuum oven with nitrogen bleed overnight at 50° C. The product was obtained as a white solid (19.75 g, 52% yield). High-resolution mass spectrometry calculated for C₁₆H₁₆NO₂: 254.1193 (M+H)⁺, found 254.1181.

Step 2 (alternate procedure): Preparation of 4,5-Dihydro-5-methyl-3,4-diphenyl-5-isoxazolol, 2.

To a 500 mL jacketed reactor equipped with a mechanical stirrer, thermocouple, and positive pressure nitrogen inlet is charged 1,2-diphenyl-ethanone, oxime (31.4 grams). Tetrahydrofuran (THF) (209 mL) is added while stirring to dissolve the solid. The reaction is cooled until a batch temperature of −15° C. is obtained. n-Hexyllithium in hexanes (131 mL, 2.3 M) is charged to the reaction vessel while keeping the temperature below 10° C. After addition is complete, the mixture is cooled down to a batch temperature of −15° C. Ethyl acetate (80 mL) is added as fast as possible. The reaction mixture is adjusted to 0° C. and then transferred to a mixture of sodium chloride (14.0 g) in water (160 mL) that is cooled to <5° C. This mixture is kept below 15° C. during the quench. The reaction vessel is rinsed with 40 mL ethyl acetate and this mixture is transferred to the quench flask. The quench mixture is warmed to 20° C. and the layers are separated. The organic layer is washed with a sodium bicarbonate (NaHCO₃) solution (9.6 g NaHCO₃/160 mL water) Toluene (120 mL) is added to the organic layer and the mixture is distilled until 67% of the pot contents are removed (temperature ˜90-93° C.). Heptane (439 mL) is added and the mixture is cooled at 0.5° C./min to 5° C. during which time crystals form. The mixture is filtered and the solid cake is washed with 100 mL of 50:50 (volume/volume) heptane:toluene. The solid is dried in a vacuum oven with nitrogen bleed overnight at 50° C. The product is obtained as a white solid (typical manufacturing yield: 59%). High-resolution mass spectrometry calculated for C₁₆H₁₆NO₂: 254.1193 (M+H)⁺, found 254.1181.

Step 3: Preparation of 4-(5-Methyl-3-phenyl-4-isoxazolyl)benzenesulfonamide (valdecoxib, 1).

4,5-Dihydro-5-methyl-3,4-diphenyl-5-isoxazolol (50.0 g, 0.197 mol) was charged to a 500 mL reactor, which had been cooled to 5° C. Trifluoroacetic acid (38.3 mL, 0.496 mol) was charged with stirring to the reactor and the 35° C. solution was cooled to ˜5° C. Chlorosulfonic acid (232 g, 1.99 mol) was added slowly to control evolution of hydrogen chloride (HCl) and maintain <25° C. during the addition. The reaction solution was then heated to 60° C. and held at 60° C. for 2.5 hours. After cooling the reaction solution to 0° C. it was added slowly to a stirred 2 to 25° C. mixture of toluene (172 mL) and water (150 mL). The reactor was rinsed with a mixture of toluene (18.4 mL) and water (50 mL), which was then added to the quench mixture. The toluene layer was extracted with water (50 mL) and cooled to 0.2° C. Concentrated ammonium hydroxide (62 mL, 1.60 mol) was added slowly with cooling to maintain ˜10 to 15° C. during the addition. The mixture was warmed slowly to 35° C. and held there for ˜40 minutes. Isopropanol (240 mL) was added, and the reaction mixture was reheated to 35° C. and held at 35° C. for 90 minutes. The crystalline mixture was slowly cooled to 20° C. and the crude product was filtered, washed with isopropanol (100 mL) and water (100 mL). The wet cake was transferred to a 500 mL crystallizer and dissolved in methanol (350 mL) at ˜58° C. Water (92 mL) was added to the methanol solution and the solution was heated to ˜70° C. This solution was slowly cooled to 50° C., held for 60 minutes and then cooled to 5° C. After one hour at 5° C. the crystalline product was collected by filtration, the cake washed with 75% methanol-water (100 mL) and dried under vacuum at ˜70° C. A differential scanning calorimetry (DSC) melting point of 171 to 174 deg C. (determined at 10 degrees C./minute) was found.

### EXAMPLE 2

Preparation of N-[[4-(5-methyl-3-phenyl-4-isoxazolyl)phenyl]sulfonyl]propanamide (parecoxib, 1a).

4-(5-methyl-3-phenyl-4-isoxazolyl)benzenesulfonamide (10.0 g, 0.032 mol) and propionic anhydride (40 mL, 0.31 mol) were charged to the 500 mL reactor. The slurry was stirred and heated to 50° C. Sulfuric acid (40 □L, 0.8 mmol) was added in one portion. All the solids dissolved and the mixture warmed to 55.5° C. within a 10 minute period after the addition was completed. The reaction mixture was then heated to 80° C. and held for approximately 10 minutes. Heating was discontinued, and the mixture was allowed to cool to 50° C. and held for about 60 minutes; solid started to crystallize from the reaction mixture at about 65° C. The mixture was slowly cooled to 0° C. and was held at 0° C. for about 60 minutes. The solid was collected by vacuum filtration. The wet cake was washed with two 45-mL portions of methyl tert-butyl ether and pulled dry at ambient temperature for about 15 minutes. The solid was further dried in a vacuum oven with a nitrogen bleed at 60° C. for 18 hours to give the solid product (8.72 g 75% yield). DSC maximum endotherm for the high melting point parecoxib is 168.95. DSC maximum endotherm for the low melting point parecoxib is 147.44.

### EXAMPLE 3

Preparation of N-[[4-(5-methyl-3-phenyl-4-isoxazolyl)phenyl]sulfonyl]propanamide, sodium salt (parecoxib sodium, 1b).

N-[[4-(5-methyl-3-phenyl-4-isoxazolyl)phenyl]-sulfonyl]propanamide (10.0 g, 0.026 mol) and 160 ml of absolute ethanol were charged to a 500 mL reactor. The slurry was heated to 45° C. and held for 30 minutes and a solution of approximately 5 weight percent sodium hydroxide in ethanol (22.4 g, 0.028 mol) was added to the reaction vessel at 45° C. After addition was completed, the solution was seeded with N-[[4-(5-methyl-3-phenyl-4-isoxazolyl)phenyl]-sulfonyl]propanamide, sodium salt, to initiate crystallization. The temperature of the reaction mixture was raised to 50° C. and held for 30 min. The mixture was slowly cooled to 0° C. and held for about 60 min. The solid was collected by vacuum filtration. The wet cake was washed twice with two 20-mL portions of absolute ethanol and was pulled dry under house vacuum with a purge of nitrogen. The solid was further dried in a vacuum oven with the nitrogen bleed at 120° C. overnight to give the solid product (9.11 g, 85% yield). DSC maximum endotherm for the form I parecoxib sodium is 274.28° C.

### EXAMPLE 4

Preparation of 5-methyl-3,4-diphenyl isoxazole, 3

4,5-dihydro-5-methyl-3,4-diphenyl-5-isoxazolol (15.0 grams, 0.059 mol) was charged to a 250 mL flask. Trifluoroacetic acid (10.5 mL) was added with stirring, and an exotherm to 44° C. was observed. The solution was heated between 44 and 57° C. for 60 minutes, cooled to room temperature, and vacuum distilled to remove trifluoroacetic acid. The residue was dissolved in 100 mL of toluene and vacuum distilled. The process was repeated a second time to provide a semi-crystalline concentrate. The concentrate was dissolved in 250 mL of hot heptane, decanted into a 500 mL flask, cooled to room temperature and held for 18 hours. The crystalline cake was broken up and the crystals were collected by filtration. The cake was dried to provide 10.19 g (73 wt % yield) of the desired product. DSC melting point: 95.55-96.24° C. at 10° C./min in an unsealed pan.

### EXAMPLE 5

Preparation of 4-(5-methyl-3-phenyl-4-isoxazolyl)benzenesulfonyl chloride, 6.

4,5-dihydro-5-methyl-3,4-diphenyl-5-isoxazolol (13.0 grams, 0.0513 mol) was charged to a 200 mL jacketed flask which was cooled with 0.2° C. jacket fluid. Trifluoroacetic acid (9.1 mL, 0.118 mol) was charged to the solids to provide a solution at 38.6° C. The solution was cooled to 2.1° C. and chlorosulfonic acid (34.7 mL, 0.522 mol) was added slowly while maintaining the temperature below 14° C. The solution was heated to 60° C., held for 2.5 hours, cooled to 20° C., and transferred to a 125 mL addition funnel. Toluene (52 mL) and water (52 mL) were charged to the 200 mL jacketed reactor, and cooled to 4° C. The reaction solution was then added slowly to the 200 mL jacketed reactor while maintaining the temperature below 20° C. The multi-phase mixture was warmed to 20° C., and transferred to a 250 mL separatory funnel. Toluene (50 mL) and water (10 mL) were added and the mixture was shaken. Settling of the mixture resulted in two cloudy phases. The toluene phase was washed twice with 15 mL of water, transferred to a 250 mL flask with a 20 mL toluene rinse, and vacuum distilled to 17.4 g of an oil. After initiating crystallization with a glass rod and cooling, heptane (20 mL) was added to the crystalline mass which was broken up to form a powder. The off white powder was collected by filtration. Portions of 50 mL of heptane were used to aid the transfer of solids to the filter. The cake was dried in a vacuum oven (35° C.) to provide 13.6 g (79.4 wt %) of the sulfonyl chloride as an 85:15 mixture of the para and meta isomers. HRMS Calculated for (M+1) C₁₆H₁₃NO₃Cl: 334.0305; Found (M+1): 334.0309.

### EXAMPLE 6

Preparation of 4-(5-methyl-3-phenyl-4-isoxazolyl)benzenesulfonyl chloride, 6.

5-methyl-3,4-diphenyl isoxazole (5.0 g, 0.0213 mol) was charged to a 100 mL jacketed reactor which was cooled with 0.2° C. jacket fluid. Trifluoroacetic acid (3.5 mL, 0.045 mol) was charged to the solids to provide a solution at 3° C. Chlorosulfonic acid (13.3 mL, 0.201 mol) was added slowly while maintaining the reaction temperature below 20° C. The solution was heated to 60° C. and held for 2.2 hours. The solution was then cooled to 6° C. and transferred to a 60 mL addition funnel. Toluene (20 mL) and water (20 mL) were charged to the 100 mL jacketed reactor and cooled to 6° C. The reaction solution was then added slowly to the 100 mL jacketed reactor while maintaining the temperature below 16° C. The multi-phase mixture was transferred to 125 mL separatory funnel. Toluene (20 mL) and water (5 mL) were added and the mixture was shaken. Settling of the mixture resulted in two cloudy phases. The toluene phase was washed twice with 5 mL of water, transferred to a 125 mL flask with a 17 mL toluene rinse, and vacuum distilled to a semi-crystalline concentrate. The concentrate was dissolved in 100 mL of toluene and vacuum distilled to an oil. After initiating crystallization with a glass rod, heptane (11 mL) was added, and the mass broken up to produce an off white powder. The solids were collected by filtration. Portions of 25 mL of heptane were used to aid the transfer of solids to the filter. The cake was dried to provide 7.07 g (100 wt %) of the sulfonyl chloride as an 85:15 mixture of the para and meta isomers. HRMS Calculated for (M+1) C₁₆H₁₃NO₃Cl: 334.0305; Found: (M+1): 334.0299.

### EXAMPLE 7

Preparation of 4-(5-Methyl-3-phenyl-4-isoxazole)benzenesulfonic Acid.

4-(5-Methyl-3-phenyl-isoxazole)benzenesulfonyl chloride (39.6 grams, 0.11 mol), water (99.5 mL, 5.5 mol) and tetrahydrofuran (558 mL) were charged to a 1-liter flask and heated to reflux overnight. After cooling to ambient temperature, the solvents were removed under pressure. The residual yellow oil was further dried under high vacuum. The resulting solid was covered with toluene (500 mL) and heated to reflux. After about 30 minutes, the solid melted and collected at the bottom of the flask. The mixture was stirred at reflux temperature for 4 hours, cooled to room temperature and stirred overnight. The solids were collected by filtration, briefly air dried and ground to a powder. The powder was suspended in toluene (500 mL), heated to reflux temperature and resolidified during the cool down to room temperature. The solids were collected by filtration and dried giving 23.8 grams of product with a melting point of 174-176° C.

What is claimed is:

1. A method of preparing an [isoxazol-4-yl]benzenesulfonamide compound having the structure of Formula 1: 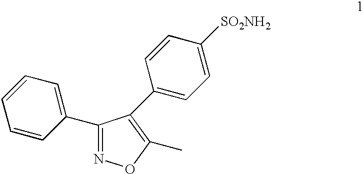
comprising:
contacting a precursor compound selected from the group consisting of Formula 2 and Formula 3: 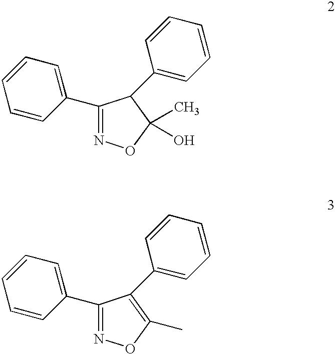
with a halosulfonic acid in the presence of trifluoroacetic acid to produce a halosulfonated product;
and contacting the halosulfonated product with a source of ammonia to produce the [isoxazol-4-yl]benzenesulfonamide compound having the structure of Formula 1.

2. The method of claim 1 wherein the halosulfonic acid is selected from the group consisting of bromosulfonic acid and chlorosulfonic acid.

3. The method of claim 1 wherein the halosulfonic acid is chlorosulfonic acid.

4. The method of claim 1 wherein the source of ammonia is selected from the group consisting of ammonium hydroxide and anhydrous ammonia.

5. The method of claim 1 wherein the source of ammonia is ammonium hydroxide.

6. The method of claim 1 wherein the source of ammonia is anhydrous ammonia.

7. A method of preparing an N-[[4-(3-phenylisoxazol-4-yl)phenyl]sulfonyl]propanamide compound having the structure of Formula 1a: 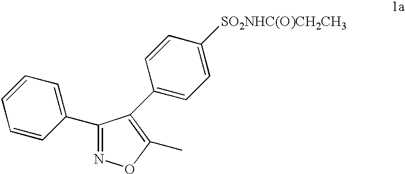
comprising:
contacting a precursor compound selected from the group consisting of Formula 2 and Formula 3: 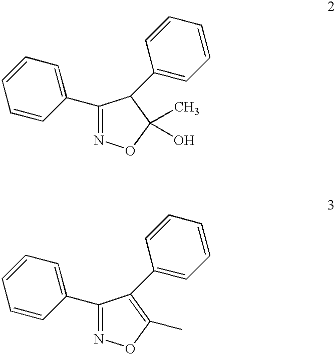
with a halosulfonic acid in the presence of trifluoroacetic acid to produce a halosulfonated product;
contacting the halosulfonated product with a source of ammonia to produce an [isoxazol-4-yl]benzenesulfonamide compound having the structure of Formula 1: 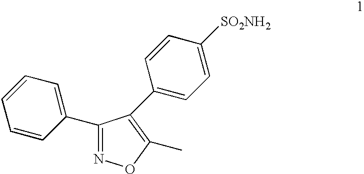
and contacting the [isoxazol-4-yl]benzenesulfonamide compound with a propionating agent to produce an N-[[4-(3-phenylisoxazol-4-yl)phenyl]sulfonyl]propanamide compound having the structure of Formula 1a.

8. The method of claim 7 wherein the halosulfonic acid is selected from the group consisting of bromosulfonic acid and chlorosulfonic acid.

9. The method of claim 7 wherein the halosulfonic acid is chlorosulfonic acid.

10. The method of claim 7 wherein the source of ammonia is selected from the group consisting of ammonium hydroxide and anhydrous ammonia.

11. The method of claim 7 wherein the source of ammonia is ammonium hydroxide.

12. The method of claim 7 wherein the source of ammonia is anhydrous ammonia.

13. The method of claim 7 wherein the propionating agent selected from the group consisting of an anhydride of propionic acid, a propionyl halide, a propionyl thioester, a propionyl carbonate and a N-propionyl imidazole.

14. The method of claim 13 wherein the propionating agent is a propionyl halide.

15. The method of claim 14 wherein the propionating agent is a propionyl chloride.

16. The method of claim 13 wherein the propionating agent is an anhydride of propionic acid.

17. The method of claim 13 wherein the anhydride of propionic acid is propionic anhydride.

18. A method of preparing an N-[[4-(3-phenylisoxazol-4-yl)phenyl]sulfonyl]propanamide, sodium salt compound having the structure of Formula 1b: 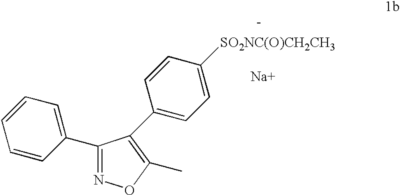
comprising:
contacting a precursor compound selected from the group consisting of Formula 2 and Formula 3: 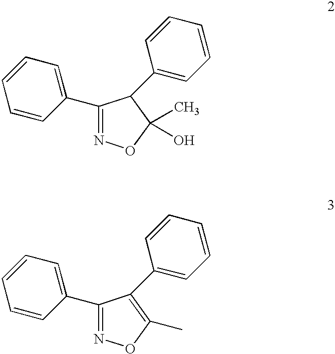
with a halosulfonic acid in the presence of trifluoroacetic acid to produce a halosulfonated product;
contacting the halosulfonated product with a source of ammonia to produce an [isoxazol-4-yl]benzenesulfonamide compound having the structure of Formula 1: 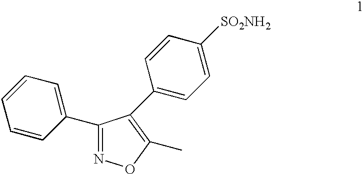
and contacting the [isoxazol-4-yl]benzenesulfonamide compound having the structure of Formula 1 with a propionating agent to produce an N-[[4-(3-phenylisoxazol-4-yl)phenyl]sulfonyl]propanamide compound having the structure of Formula 1a: 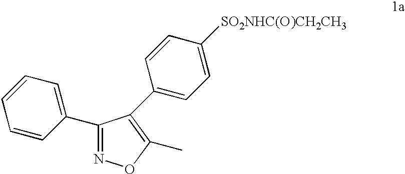
and further contacting the compound of Formula 1a with a sodium base to produce an N-[[4-(3-phenylisoxazol-4-yl)phenyl]sulfonyl]propanamide, sodium salt compound having the structure of Formula 1b.

19. The method of claim 18 wherein the halosulfonic acid is selected from the group consisting of bromosulfonic acid and chlorosulfonic acid.

20. The method of claim 18 wherein the halosulfonic acid is chlorosulfonic acid.

21. The method of claim 18 wherein the source of ammonia is selected from the group consisting of ammonium hydroxide and anhydrous ammonia.

22. The method of claim 18 wherein the source of ammonia is ammonium hydroxide.

23. The method of claim 18 wherein the source of ammonia is anhydrous ammonia.

24. The method of claim 18 wherein the propionating agent is selected from the group consisting of an anhydride of propionic acid, a propionyl halide, a propionyl thioester, a propionyl carbonate and a N-propionylimidazole.

25. The method of claim 24 wherein the propionating agent is a propionyl halide.

26. The method of claim 25 wherein the propionating agent is a propionyl chloride.

27. The method of claim 24 wherein the propionating agent is an anhydride of propionic acid.

28. The method of claim 24 wherein the anhydride of propionic acid is propionic anhydride.

29. The method of claim 18 wherein the sodium base is selected from the group consisting of sodium hydroxide, a sodium alkoxide, sodium hydride and sodium carbonate.

30. The method of claim 29 wherein the sodium base is sodium hydroxide.

31. A method of preparing an [isoxazol-4-yl]benzenesulfonamide compound having the structure of Formula 1: 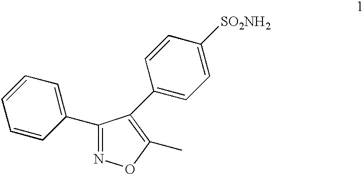
comprising:
forming a diphenylethanone oxime derivative compound by contacting a 1,2-diphenylethanone compound with a source of hydroxylamine;
contacting the oxime derivative compound with a strong base and an acetylating agent to form a diphenylisoxazoline derivative;
contacting the diphenylisoxazoline derivative with trifluoroacetic acid and a halosulfonic acid to form a halosulfonated product;
and contacting the halosulfonated product with a source of ammonia to produce an [isoxazol-4-yl]benzenesulfonamide compound having the structure of Formula 1.

32. The method of claim 31 wherein the source of hydroxylamine is an aqueous solution comprising hydroxylamine.

33. The method of claim 31 wherein the source of hydroxylamine is an aqueous solution comprising hydroxylamine and a weak acid.

34. The method of claim 33 wherein the weak acid is a carboxylic acid.

35. The method of claim 33 wherein the carboxylic acid is an alkyl carboxylic acid.

36. The method of claim 33 wherein the alkyl carboxylic acid selected from the group consisting of formic acid, acetic acid and propionic acid.

37. The method of claim 33 wherein the alkyl carboxylic acid is acetic acid.

38. The method of claim 31 wherein the source of hydroxylamine is an aqueous solution comprising hydroxylamine and a conjugate base of a weak acid.

39. The method of claim 38 wherein the conjugate base of a weak acid is sodium acetate.

40. The method of claim 31 wherein the source of hydroxylamine comprises a hydroxylamine salt and a deprotonating base.

41. The method of claim 40 wherein the hydroxylamine salt is selected from a group consisting of hydroxylamine hydrochloride, hydroxylamine sulfate and hydroxylamine acetate.

42. The method of claim 41 wherein the hydroxylamine salt is hydroxylamine hydrochloride.

43. The method of claim 40 wherein the deprotonating base is selected from the group consisting of sodium hydroxide, potassium hydroxide, and sodium acetate.

44. The method of claim 40 wherein the deprotonating base is sodium acetate.

45. The method of claim 40 wherein the source of hydroxylamine comprises hydroxylamine and acetic acid.

46. The method of claim 31 wherein the strong base is selected from the group consisting of a lithium dialkylamide, an aryl lithium, an arylalkyl lithium and an alkyl lithium.

47. The method of claim 31 wherein the strong base is a lithium dialkylamide.

48. The method of claim 47 wherein the strong base is lithium diisopropylamide.

49. The method of 46 wherein the strong base is a C₁ to about C10 alkyl lithium.

50. The method of claim 31 wherein the strong base is butyl lithium.

51. The method of claim 31 wherein the strong base is hexyl lithium.

52. The method of claim 31 wherein the strong base is heptyl lithium.

53. The method of claim 31 wherein the strong base is octyl lithium.

54. The method of claim 31 wherein the acetylating agent is selected from the group consisting of an alkyl acetate, an acetic anhydride, an N-alkyl-N-alkoxyacetamide and an acetyl halide.

55. The method of claim 54 wherein the acetylating agent is a C1 to about C6 alkyl acetate.

56. The method of claim 31 wherein the acetylating agent is selected from the group consisting of methyl acetate, ethyl acetate, propyl acetate and butyl acetate.

57. The method of claim 56 wherein the alkyl acetate is ethyl acetate.

58. The method of claim 31 wherein the acetylating agent is an acetyl halide.

59. The method of claim 58 wherein the acetyl halide is acetyl chloride.

60. The method of claim 31 wherein the acetylating agent is acetic anhydride.

61. The method of claim 31 wherein the halosulfonic acid is selected from the group consisting of bromosulfonic acid and chlorosulfonic acid.

62. The method of claim 31 wherein the halosulfonic acid is chlorosulfonic acid.

63. The method of claim 31 wherein the source of ammonia is selected from the group consisting of ammonium hydroxide and anhydrous ammonia.

64. The method of claim 31 wherein the source of ammonia is ammonium hydroxide.

65. The method of claim 31 wherein the source of ammonia is anhydrous ammonia.

66. A method of preparing an N-[[4-(3-phenylisoxazol-4-yl)phenyl]sulfonyl]propanamide compound having the structure of Formula 1a: 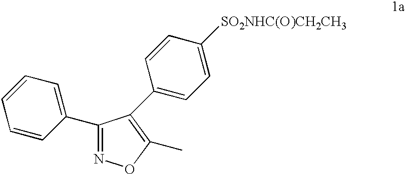
comprising:
forming a diphenylethanone oxime derivative by contacting a 1,2-diphenylethanone compound with a source of hydroxylamine;
contacting the oxime with a strong base and an acetylating agent to form a diphenylisoxazoline derivative;
contacting the diphenylisoxazoline derivative with trifluoroacetic acid and a halosulfonic acid to form a halosulfonated product;
contacting the halosulfonated product with a source of ammonia to produce an [isoxazol-4-yl]benzenesulfonamide compound having the structure of Formula 1: 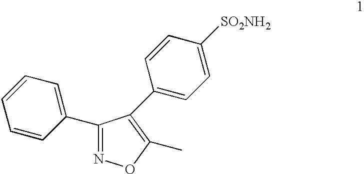
and contacting the [isoxazol-4-yl]benzenesulfonamide compound of Formula 1 with a propionating agent to produce an N-[[4-(3-phenylisoxazol-4-yl)phenyl]sulfonyl]propanamide compound having the structure of Formula 1a.

67. The method of claim 66 wherein the source of hydroxylamine is an aqueous solution comprising hydroxylamine.

68. The method of claim 66 wherein the source of hydroxylamine is an aqueous solution comprising hydroxylamine and a weak acid.

69. The method of claim 68 wherein the weak acid is a carboxylic acid.

70. The method of claim 68 wherein the carboxylic acid is an alkyl carboxylic acid.

71. The method of claim 68 wherein the alkyl carboxylic acid selected from the group consisting of formic acid, acetic acid and propionic acid.

72. The method of claim 68 wherein the alkyl carboxylic acid is acetic acid.

73. The method of claim 66 wherein the source of hydroxylamine is an aqueous solution comprising hydroxylamine and a conjugate base of a weak acid.

74. The method of claim 73 wherein the conjugate base of a weak acid is sodium acetate.

75. The method of claim 66 wherein the source of hydroxylamine comprises a hydroxylamine salt and a deprotonating base.

76. The method of claim 75 wherein the hydroxylamine salt is selected from a group consisting of hydroxylamine hydrochloride, hydroxylamine sulfate and hydroxylamine acetate.

77. The method of claim 76 wherein the hydroxylamine salt is hydroxylamine hydrochloride.

78. The method of claim 75 wherein the deprotonating base is selected from the group consisting of sodium hydroxide, potassium hydroxide, and sodium acetate.

79. The method of claim 75 wherein the deprotonating base is sodium acetate.

80. The method of claim 75 wherein the source of hydroxylamine comprises hydroxylamine and acetic acid.

81. The method of claim 66 wherein the strong base is selected from the group consisting of a lithium dialkylamide, an aryl lithium, an arylalkyl lithium and an alkyl lithium.

82. The method of claim 66 wherein the strong base is a lithium dialkylamide.

83. The method of claim 82 wherein the strong base is lithium diisopropylamide.

84. The method of claim 81 wherein the strong base is a C₁ to about C₁₀ alkyl lithium.

85. The method of claim 66 wherein the strong base is butyl lithium.

86. The method of claim 66 wherein the strong base is hexyl lithium.

87. The method of claim 66 wherein the strong base is heptyl lithium.

88. The method of claim 66 wherein the strong base is octyl lithium.

89. The method of claim 66 wherein the acetylating agent is selected from the group consisting of an alkyl acetate, an acetic anhydride, an N-alkyl-N-alkoxyacetamide and an acetyl halide.

90. The method of claim 89 wherein the acetylating agent is a C1 to about C6 alkyl acetate.

91. The method of claim 66 wherein the acetylating agent is selected from the group consisting of methyl acetate, ethyl acetate, propyl acetate and butyl acetate.

92. The method of claim 91 wherein the alkyl acetate is ethyl acetate.

93. The method of claim 66 wherein the acetylating agent is an acetyl halide.

94. The method of claim 93 wherein the acetyl halide is acetyl chloride.

95. The method of claim 66 wherein the acetylating agent is acetic anhydride.

96. The method of claim 66 wherein the halosulfonic acid is selected from the group consisting of bromosulfonic acid and chlorosulfonic acid.

97. The method of claim 66 wherein the halosulfonic acid is chlorosulfonic acid.

98. The method of claim 66 wherein the source of ammonia is selected from the group consisting of ammonium hydroxide and anhydrous ammonia.

99. The method of claim 66 wherein the source of ammonia is ammonium hydroxide.

100. The method of claim 66 wherein the source of ammonia is anhydrous ammonia.

101. The method of claim 66 wherein the propionating agent selected from the group consisting of an anhydride of propionic acid, a propionyl halide, a propionyl thioester, a propionyl carbonate and a N-propionyl imidazole.

102. The method of claim 101 wherein the propionating agent is a propionyl halide.

103. The method of claim 102 wherein the propionating agent is a propionyl chloride.

104. The method of claim 101 wherein the propionating agent is an anhydride of propionic acid.

105. The method of claim 104 wherein the anhydride of propionic acid is propionic anhydride.

106. A method of preparing an N-[[4-(3-phenylisoxazol-4-yl)phenyl]sulfonyl]propanamide, sodium salt compound having the structure of Formula 1b: 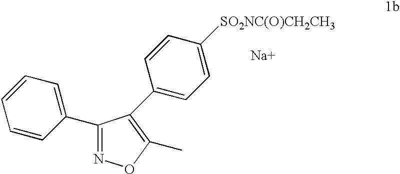
comprising:
forming a diphenylethanone oxime derivative by contacting a 1,2-diphenylethanone compound with a source of hydroxylamine;
contacting the oxime compound with a strong base and an acetylating agent to form a diphenylisoxazoline derivative;
contacting the diphenylisoxazoline derivative with trifluoroacetic acid and a halosulfonic acid to form a halosulfonated product;
contacting the halosulfonated product with a source of ammonia to produce an [isoxazol-4-yl]benzenesulfonamide compound having the structure of Formula 1: 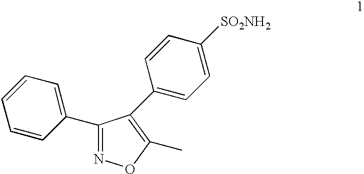
contacting an [isoxazol-4-yl]benzenesulfonamide compound with a propionating agent to produce an N-[[4-(3-phenylisoxazol-4-yl)phenyl]sulfonyl]propanamide compound having the structure of Formula 1a: 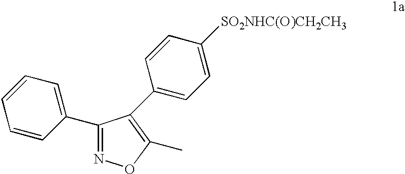
and contacting the N-[[4-(3-phenylisoxazol-4-yl)phenyl]sulfonyl]propanamide compound with a sodium base to produce a N-[[4-(3-phenylisoxazol-4-yl)phenyl]sulfonyl]propanamide, sodium salt compound having the structure of Formula 1b.

107. The method of claim 106 wherein the source of hydroxylamine is an aqueous solution comprising hydroxylamine.

108. The method of claim 106 wherein the source of hydroxylamine is an aqueous solution comprising hydroxylamine and a weak acid.

109. The method of claim 108 wherein the weak acid is a carboxylic acid.

110. The method of claim 108 wherein the carboxylic acid is an alkyl carboxylic acid.

111. The method of claim 108 wherein the alkyl carboxylic acid selected from the group consisting of formic acid, acetic acid and propionic acid.

112. The method of claim 108 wherein the alkyl carboxylic acid is acetic acid.

113. The method of claim 106 wherein the source of hydroxylamine is an aqueous solution comprising hydroxylamine and a conjugate base of a weak acid.

114. The method of claim 113 wherein the conjugate base of a weak acid is sodium acetate.

115. The method of claim 106 wherein the source of hydroxylamine comprises hydroxylamine salt and a deprotonating base.

116. The method of claim 106 wherein the hydroxylamine salt is selected from a group consisting of hydroxylamine hydrochloride, hydroxylamine sulfate and hydroxylamine acetate.

117. The method of claim 116 wherein the hydroxylamine salt is hydroxylamine hydrochloride.

118. The method of claim 115 wherein the deprotonating base is selected from the group consisting of sodium hydroxide, potassium hydroxide, and sodium acetate.

119. The method of claim 115 wherein the deprotonating base is sodium acetate.

120. The method of claim 115 wherein the source of hydroxylamine comprises hydroxylamine and acetic acid.

121. The method of claim 106 wherein the strong base is selected from the group consisting of a lithium dialkylamide, an aryl lithium, an arylalkyl lithium and an alkyl lithium.

122. The method of claim 106 wherein the strong base is a lithium dialkylamide.

123. The method of claim 122 wherein the strong base is lithium diisopropylamide.

124. The method of 121 wherein the strong base is a C₁ to about C₁₀ alkyl lithium.

125. The method of claim 106 wherein the strong base is butyl lithium.

126. The method of claim 106 wherein the strong base is hexyl lithium.

127. The method of claim 106 wherein the strong base is heptyl lithium.

128. The method of claim 106 wherein the strong base is octyl lithium.

129. The method of claim 106 wherein the acetylating agent is selected from the group consisting of an alkyl acetate, an acetic anhydride, an N-alkyl-N-alkoxyacetamide and an acetyl halide.

130. The method of claim 240 wherein the acetylating agent is a C1 to about C6 alkyl acetate.

131. The method of claim 106 wherein the acetylating agent is selected from the group consisting of methyl acetate, ethyl acetate, propyl acetate and butyl acetate.

132. The method of claim 131 wherein the alkyl acetate is ethyl acetate.

133. The method of claim 106 wherein the acetylating agent is an acetyl halide.

134. The method of claim 133 wherein the acetyl halide is acetyl chloride.

135. The method of claim 106 wherein the acetylating agent is acetic anhydride.

136. The method of claim 106 wherein the halosulfonic acid is selected from the group consisting of bromosulfonic acid and chlorosulfonic acid.

137. The method of claim 106 wherein the halosulfonic acid is chlorosulfonic acid.

138. The method of claim 106 wherein the source of ammonia is selected from the group consisting of ammonium hydroxide and anhydrous ammonia.

139. The method of claim 106 wherein the source of ammonia is ammonium hydroxide.

140. The method of claim 106 wherein the source of ammonia is anhydrous ammonia.

141. The method of claim 106 wherein the propionating agent selected from the group consisting of an anhydride of propionic acid, a propionyl halide, a propionyl thioester, a propionyl carbonate and a N-propionyl imidazole.

142. The method of claim 141 wherein the propionating agent is a propionyl halide.

143. The method of claim 142 wherein the propionating agent is a propionyl chloride.

144. The method of claim 141 wherein the propionating agent is an anhydride of propionic acid.

145. The method of claim 144 wherein the anhydride of propionic acid is propionic anhydride.

146. The method of claim 106 wherein the sodium base is selected from the group consisting of sodium hydroxide, a sodium alkoxide, sodium hydride and sodium carbonate.

147. The method of claim 146 wherein the sodium base is sodium hydroxide.

148. A method of preparing a benzenesulfonyl halide compound having the structure of Formula 4 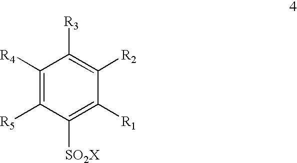
wherein:
X is a halogen atom and R¹, R², R³, R⁴ and R⁵ are each independently selected from the group consisting of hydrogen, alkyl, alkenyl, alkynyl, cycloalkyl, aryl, heterocyclyl, alkoxy, alkylamino, alkylthio and acyl; wherein alkyl, alkenyl, cycloalkyl, aryl, heterocyclyl is each independently optionally substituted with one or more moieties selected from the group consisting of alkyl, alkenyl, alkynyl, cycloalkyl, alkylaryl, aryl, heterocyclyl, alkoxy, alkylamino, alkylthio, acyl, halo, haloalkylaryl, alkoxyaryl, haloalkyl, protected hydroxymethyl, arylalkoxymethyl and alkoxyhaloalkyl; wherein the method comprises contacting a substituted phenyl compound having the structure of Formula 5: 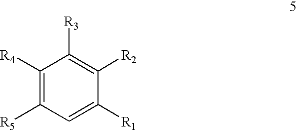
with a halosulfonic acid in the presence of trifluoroacetic acid, thereby forming a benzenesulfonyl halide compound having the structure of Formula 4.

149. A method of claim 148 wherein the halosulfonic acid is selected from the group consisting of bromosulfonic acid and chlorosulfonic acid.

150. A method of claim 148 wherein the halosulfonic acid is chlorosulfonic acid.

151. A method of claim 148 wherein R³ is heterocyclyl optionally substituted with one or more moieties selected from the group consisting of alkyl, alkenyl, alkynyl, cycloalkyl, alkylaryl, aryl, heterocyclyl, alkoxy, alkylamino, alkylthio, acyl, halo, haloalkylaryl, alkoxyaryl, haloalkyl, protected hydroxymethyl, arylalkoxymethyl and alkoxyhaloalkyl; and R¹, R², R⁴ and R⁵ are hydrogen.

152. A method of claim 151 wherein R³ is selected from the group consisting of isoxazolyl and pyrazolyl, wherein R³ is optionally substituted with one or more moieties selected from the group consisting of alkyl, alkenyl, alkynyl, cycloalkyl, alkylaryl, aryl, heterocyclyl, alkoxy, alkylamino, alkylthio, acyl, halo, haloalkylaryl, alkoxyaryl, haloalkyl, protected hydroxymethyl, arylalkoxymethyl and alkoxyhaloalkyl; and R¹, R², R⁴ and R⁵ are hydrogen.

153. A method of claim 152 wherein the benzenesulfonyl halide compound is 4-[5-methyl-3-phenylsioxazol-4-yl]benzenesulfonyl chloride compound having the structure of Formula 6: [chemical structure]
